(19) United States
(12) Patent Application Publication
(10) Pub. No.: US 2014/0249536 A1
(43) Pub. Date: Sep. 4, 2014

(54) DISTAL LOCKING TARGETING DEVICE

(71) Applicant: Levant Orthopaedics Limited, Brierley Hill, West Midlands (GB)

(72) Inventor: Salah Eddin Jajeh, Brierley Hill (GB)

(21) Appl. No.: 14/350,006

(22) PCT Filed: Oct. 8, 2012
(86) PCT No.: PCT/GB2012/052488

§ 371 Date: Apr. 4, 2014

(30) Foreign Application Priority Data
Oct. 7, 2011 (GB) 1117370.5

(51) Int. Cl. A61B 17/17; A61B 17/72

(52) U.S. Cl. CPC A61B 17/1725; A61B 17/72
USPC 606/96

(57) ABSTRACT

Devices and methods for locating apertures in a surgical nail inserted in a patient. The apertures in the surgical nail may be positionally calibrated relative to disclosed devices prior to insertion in a bone of the patient, with the calibration used to locate the apertures in the surgical nail after insertion in the bone of the patient.

This invention relates generally to distal locking of surgical nails and more particularly but not exclusively to so-called an IntraMedullary Nail (IMN), used to fix long bone fractures.

Distal locking is recommended for unstable fractures and when rotational stability is required. Most surgeons currently use fluoroscope imaging to help guide the process of drilling holes for screws in the distal end of a bone within which a nail is to be secured. Fluoroscopy, while essential for proper nail placement can subject the surgical team and patient to substantial radiation. Improved targeting and placement would reduce such exposure.

The standard operating procedure for the fixation of long bone fractures with an IntraMedullary Nail (IMN) consists of the implantation of the IMN, an elongated, rod-like, in the bone. The surgeon typically starts with an incision and thereafter creates an opening in the proximal or distal end (antegrade or retrograde) of a given long bone, thereby providing access to the affected medullary canal in which the IMN is then implanted. One goal in IMN treatment is fracture reduction so that near anatomic alignment takes place under stabilized conditions, often facilitated by means of locking screws that are integrated within the IMN.

Any given IMN is typically prefabricated, for example, with locking holes that are located in the general vicinity of the proximal and distal ends of the IMN. There are several holes at each end of the nail and each hole takes one screw. The locking screws when interlocked with an IMN are designed to stabilize the fracture and fix the bone relative to the implanted IMN.

In order to accurately lock long intramedullary nails the distal screws have to be accurately aligned with the holes in the nail. This locking is complicated by the deflection of the nail during insertion into the bone canal which normally changes the location of the hole from its initial ‘relaxed’ position before insertion. In such circumstances the surgeon has been forced in the past to determine hole positions in the nail freehand with the help of an X-ray C-arm. A common problem in such a procedure is that the surgical instruments are “in the way” since they are on the image plane of the C-arm, obstructing a clear view, the procedure also takes a long time and exposes the surgical team to excessive radiation.

Several simplified techniques, jigs, laser assisted and mechanical guiding instruments, and surgical navigation systems have been used to perform distal interlocking of intramedullary nails, but none has found widespread acceptance. Mechanical aiming systems had been introduced for radiation independent tibial and femoral nail interlocking, but they were not successful because of failure to take into account the deformation the nail undergoes during insertion into the medullary cavity. Secondly, the manufacturers can't guarantee the nails to have exactly the same shape as minor differences could happen during the manufacturing process.

In accordance with aspects of the first invention there is provided a locator for a surgical nail, the locator comprising a projection block for surgical pins and lockable upon a positioning arm spaced in use relative to a surgical nail, the positioning block having an arch to extend in use towards a surgical nail, the arch having a guide and a rod guided by the guide, the rod having a contact end to engage the surgical nail when projected through the guide and marking means to indicate the rod position upon engagement with the surgical nail for subsequent return of relative positioning between the positioning block and a surgical nail in use.

Aspects of the first invention also include a method of locating a surgical nail in a bone, the method comprising:
a) Prior to insertion of a surgical nail into a bone cavity associating the surgical nail with an arm having a positioning block adjacent a distal end of the nail;
b) Securing the block towards the distal end of the surgical nail thorough nail apertures;
c) Positioning a calibration arch with a guide over a part of the distal end of the surgical nail, placing within the guide a rod with a foot end to engage a surface of the surgical nail and marking the position of the rod upon the rod or an associated part when the foot end engages the surface of the surgical nail with a mark or recording that position;
d) Detaching the whole device from the proximal end of the nail and inserting the nail into a bone cavity;
e) Re-attaching as required the now inserted surgical nail in the bone cavity and arm as previously with the calibration arch extending over the nail;
f) Inserting the rod and providing the foot end in engagement with the surface of the surgical nail and adjusting the block to return to the previous marker position if required.

In accordance with aspects of the second invention there is provided a distal locking positioning arrangement for a surgical nail, the arrangement comprising a positioning arm associated with a universal joint with a locking cam joint, the arm has extendable telescopically means, and at a distal end there is an positioning aperture whereby in use a pin can extend through the positioning aperture to a nail aperture in a surgical nail and at least when suitably aligned the universal joint and the extension means are lockable to allow removal and return of the nail if required.

Aspects of the second invention include a method of positioning a surgical nail in a bone cavity, the method comprising:
a) Assembling a surgical nail with a positioning arrangement comprising an adjustable arm with extension means and/or a universal joint in a free state, the association being at both a proximal end as required and at distal end by fastenings between positioning apertures in a block upon the arm and nail apertures in the distal end of the nail;
b) Once assembled lock the adjustable arm to maintain orientation and configuration;
c) Releasing at least association between the block and the distal end of the surgical nail then insertion of the nail into a bone cavity;
d) Re-assembly of the nail with the locked positioning arrangement whereby the block provides through the positioning apertures a guide for a drill device in alignment with the nail apertures in the distal end of the surgical nail.

The universal joint may be a ball and socket type union or joint.

The positioning block may have a plurality of positioning apertures.

The universal joint may comprise a number of universal joint elements. The universal joints may be intermediate along the length of the arm. The positioning arm is adjustable at least along an axis in use parallel to a surgical nail.

The positioning block may comprise a carrier incorporating the positioning aperture in a frame whereby the carrier is moveable in the frame by adjustment means for subsequent return of relative positioning between the positioning block and a surgical nail in use. The adjustment means may be by a screw thread attachment or a sliding mechanism.

The guide may be a sleeve. The sleeve may extend substantially perpendicularly at a spaced position from the positioning block.

The marking means may be a semi-permanent ink. The marking means may be a clamp or similar retainer associated with the rod. The marking means may be increments marked on the rod to allow recordal of rod position relative to part of the guide. The increments may correspond with incremental adjustment by the adjustment means.

In accordance with third aspects of the present invention there is provided a distal locking positioning arrangement for a surgical nail, the arrangement comprising a positioning arm with a positioning clamp at one end and a distal end, the positioning arm articulated with a pivot between a clamp part and a distal part, a cross-member secured between the clamp part and the distal part of the positioning arm by a fastener over a positioning range to fix the pivot between the clamp part and the distal part of the positioning arm whereby the distance between the positioning clamp and the distal end can be varied and set by the fastener.

The pivot may be a hinge.

The cross-member may be respectively associated with the clamp part and the distal part at substantially the same distance from the pivot.

One end of the cross member may be position ally fixed and the fastener positionally varied on the cross-member by positional variation means. The positional variation means may be a slide or a ratchet.

The distance between the clamp end and the distal end of the positioning arm may be at a minimum in a line substantially parallel to the cross-member.

The cross-member may be incrementally marked or mark able to replicate position.

The cross-member may be straight or curved.

Aspects of the third invention include a method of positioning a surgical nail in a bone cavity, the method comprising:
a) Assembling a surgical nail with a positioning arrangement comprising a positioning arm with extension means provided by articulation about a pivot between a clamp part and a distal part, the assembly being at both a proximal end by the clamp part as required and at the distal end by fastenings between positioning apertures in a block upon the arm and nail apertures in the distal end of the nail;
b) Once assembled lock the positioning arm to maintain orientation and configuration by a cross-member;
c) Releasing at least association between the block and the distal end of the surgical nail then insertion of the nail into a bone cavity;
d) Re-assembly of the nail with the locked positioning arrangement whereby the block provides through the positioning apertures a guide for a drill device in alignment with the nail apertures in the distal end of the surgical nail.
The positioning arm is as described above with regard to aspects of the third invention.

In accordance with a fourth aspect of the present invention there is provided a target device for a surgical nail using irradiation, the target device comprising a plurality of irradiation opaque line elements and an irradiation target line element with a displacement element between them, the target device having a mounting for securing the target device to a positioning arm whereby substantially exact coincidence of the opaque line elements one upon the other in an irradiation image provided by an irradiation source to one side defines alignment of the target device and the irradiation source, the displacement element provides the target line element as a reference a clear distance away from the opaque line elements for a surgical nail in use.

The target device may be provided by a housing formed from an irradiation clear material to present the opaque line elements and the target line element from the displacement element.

The opaque line elements and/or the target line element may be formed from wire. The opaque line elements may be continuous and solid. The opaque line elements may be formed from segments to provide a broken line. The opaque line elements may be straight or wavy. The opaque line elements may have uprights. The uprights may be at the ends. The opaque line elements may be parallel and separated in a plane relative to the target line.

The target line element may be straight or shaped to interact with parts such as apertures of a surgical nail in use.

The mounting may be spaced pegs.

Also in accordance with fourth aspects of the present invention there is provided a method of positioning a surgical nail in a bone cavity, the method comprising:
a) Assembling a surgical nail with a positioning arrangement comprising a positioning arm with adjustment means, the association being at both a proximal end as required and at a distal end by fastenings between positioning apertures in a block upon the arm and nail apertures in the distal end of the nail;
b) Once assembled lock the adjustable arm to maintain orientation and configuration;
c) Releasing at least association between the block and the distal end of the surgical nail then insertion of the nail into a bone cavity;
d) Associate a target device as described above with the distal end of the positioning arm and locate an irradiation source to one side and an irradiated image device to the other whereby the positioning arm through the opaque line element overlap is confirmed as parallel in a plane between the source and the image device;
e) Using the target line element as a reference adjust the positioning arm until the reference coincides with a feature of the nail inserted into the bone cavity then locking the positioning element.
f) Re-assembly of the positioning arrangement with the block so that the block provides through the positioning apertures a guide for a drill device in alignment with the nail apertures in the distal end of the surgical nail.

Embodiments of the first invention, the second invention, the third invention and the fourth invention will now be described by way of example only with reference to the accompanying drawings in which:

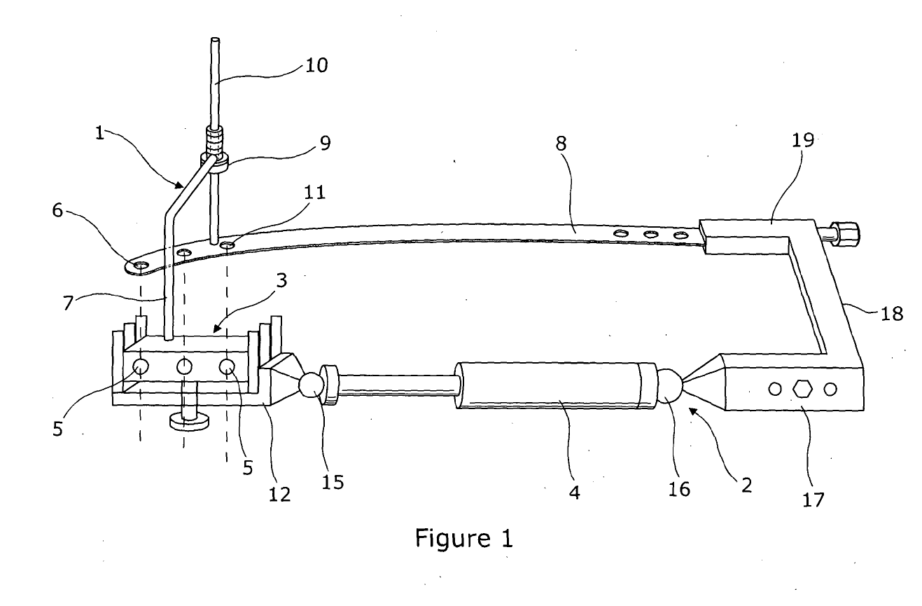

FIG. 1 is a schematic perspective view of both first and second inventions in accordance with aspects of the present invention;

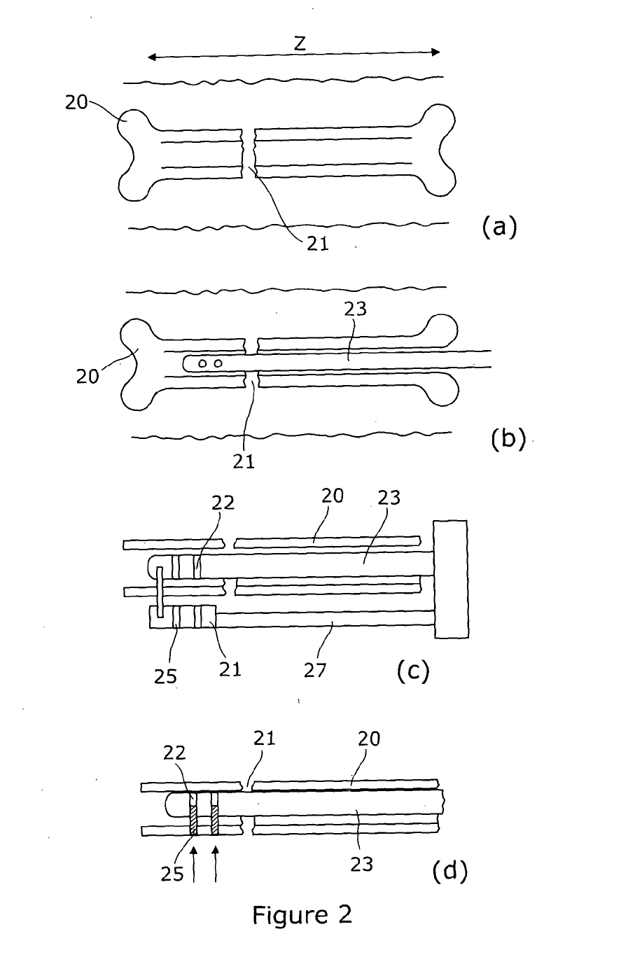

FIG. 2 is a schematic illustration of stages a) to d) of inserting, positioning and locking a surgical nail in accordance with aspects of the present invention;

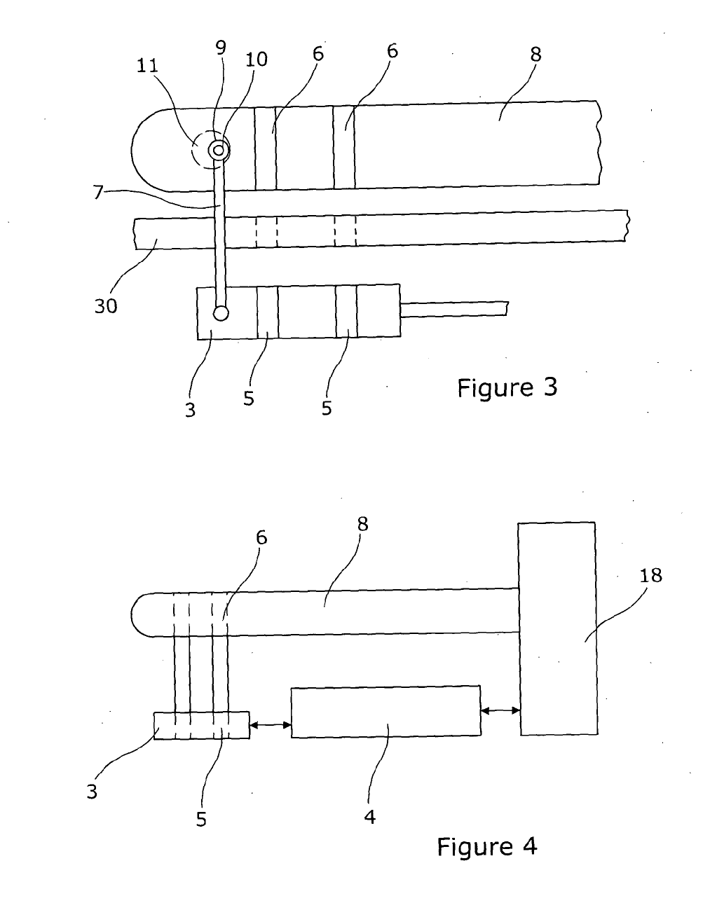

FIG. 3 is a schematic illustration of a first invention in accordance with aspects of the present invention;

FIG. 4 is a schematic illustration of a second invention in accordance with aspects of the present invention;

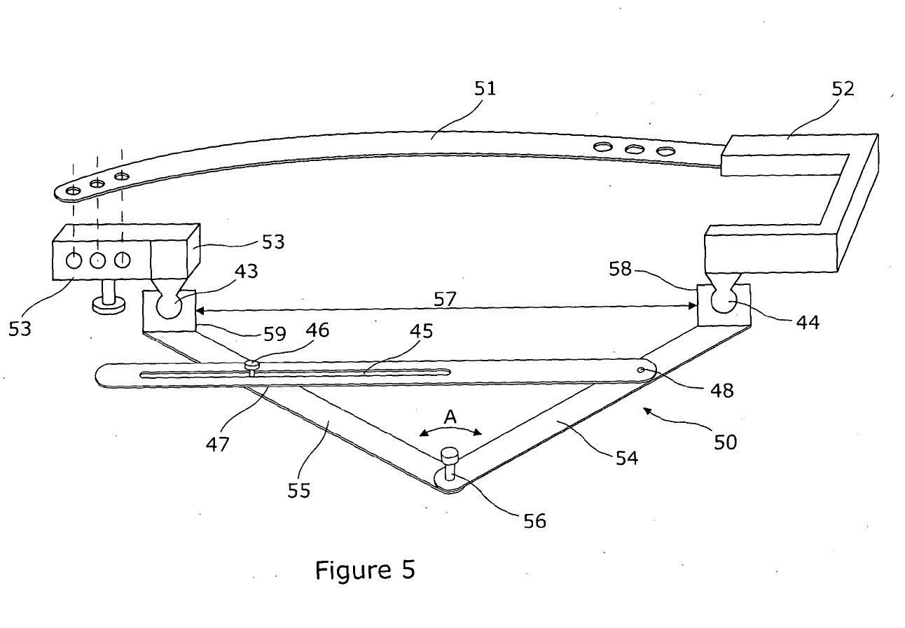

FIG. 5 is a schematic illustration of a third invention in accordance with aspects of the present invention;

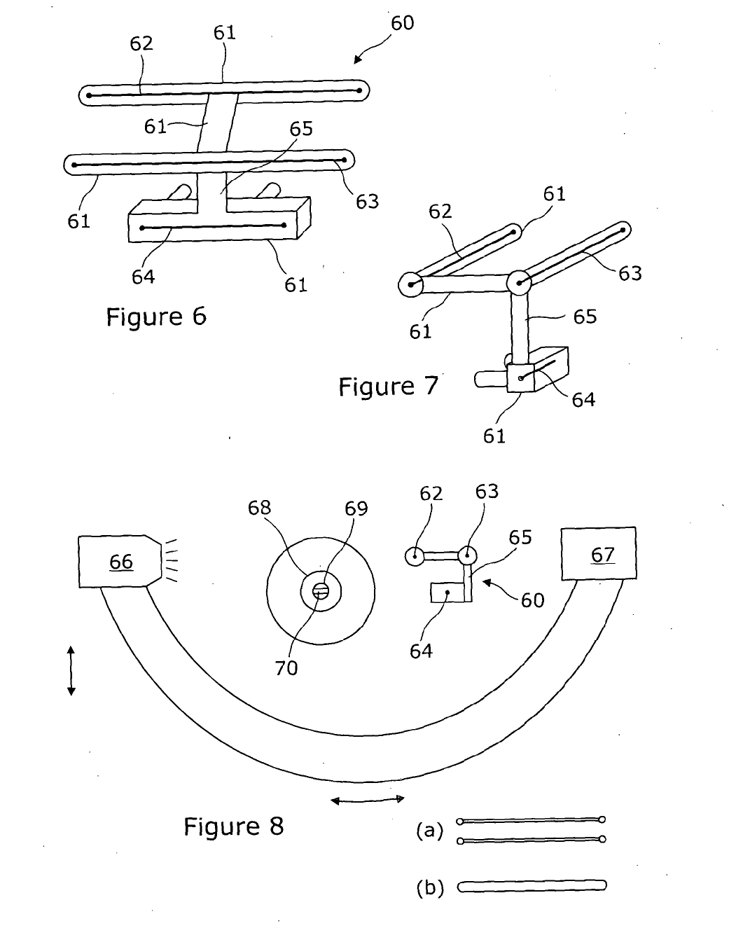

FIG. 6 is a schematic upper perspective illustration of a fourth invention in accordance with aspects of the present invention;

FIG. 7 is a schematic front perspective illustration of the fourth invention as depicted in FIG. 6;

FIG. 8 is a schematic illustration of the fourth invention depicted in FIG. 6 and FIG. 7 in use;

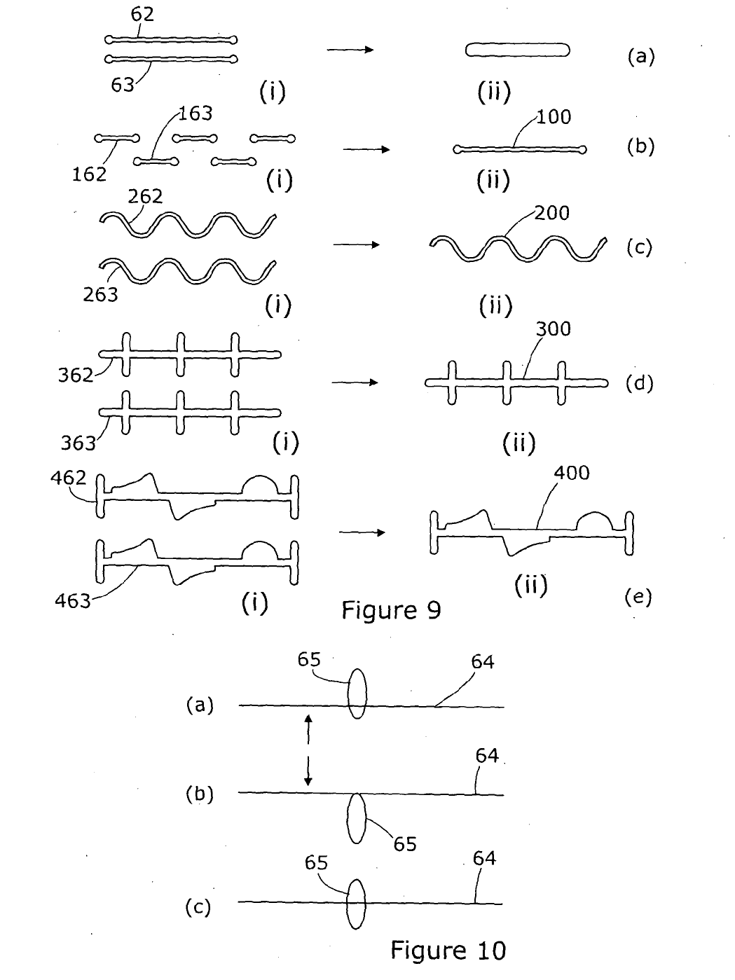

FIG. 9 provides schematic illustrations a) to e) of example possible opaque line elements in accordance with the fourth invention; and,

FIG. 10 provides schematic illustrations a) to c) of a target line in accordance with aspects of the present invention used with a surgical nail.

As indicated above positioning of a surgical nail is important to provide effective stabilization of a fracture, but with an IntraMedullary Nail there are the added problems of the surgeon working blind in terms of accurate actual screw or nail aperture position within the bone. It will be understood that the nail is located within the bone so not only are there manufacturing tolerance variations but also distortions of the nail as it is driven into the bone during the procedure. As indicated above previously Fluoroscopy, free hand techniques and surgeon skill have been depended upon to ensure that an accurately drilled hole in the bone aligns with a hole in the nail itself so that a screw can then be located as required. Correct location of the drilled aperture in the bone is necessary to avoid weakening of the bone with a bigger aperture than necessary and to fully stabilize the nail against slippage which may lead to premature failure.

Aspects of the present inventions respectively relate to proximal positioning of a surgical nail and positional correction for accurate positioning and locking with screws as required. A first invention provides a locator which includes an arch to extend over to a surgical nail initially outside of a bone and then with the nail in position within a bone so that a marked rod within a guide can ensure subsequent return to relative positioning between a projection block for a drill and a pin or nail aperture position in a nail within a bone. A second invention relates to providing a distal lock positioning arrangement which includes a positioning arm which has universal adjustment through a joint and extension between a mounting end and a distal end upon which a positioning block is located so that a pin can be arranged to extend between the positioning block and the nail aperture so that the positioning arm can then be locked, the pin removed then the nail inserted into a bone and the position block used to drill an aperture in the bone to the nail aperture in the surgical nail now located in the bone to allow a screw be secured to stablise the distal end of the surgical nail in use. A third invention relates to an alternative means of providing a least length adjustment of a positioning arm. A fourth invention relates to a target device for location of positioning arm and/or a surgical nail such as an IMN.

Advantages associated with the usage of an IMN include providing sufficient stability to maintain alignment and length, and limit rotation of the fracture site. Further advantages include allowing minimally invasive techniques, reduced hospitalization, earlier postoperative ambulation, and an earlier active range of motion (ROM) when compared to some conventional casting or external fixation methods.

All inventions individually and when combined in one device will allow better positioning and locking of a surgical nail with reduction of the X-Ray exposure and operating theatre time due to quicker correct positioning and location of the locking nail aperture or hole along with typically easier and more surety of position in use.

Aspects of the inventions allow for adjustment due to deformation of the surgical nail during installation within a bone. Such adjustment may potentially reduce a need for intuitive skill level requirements of the surgeon to be built up with experience and training so benefiting operating times and also the need for a specialist radiographer. Furthermore some equipment which may inhibit access by the surgeon such as an image intensifier will not be required and the inventions could be used with both solid and cannulated surgical nails. The inventions are adjustable and adaptable to almost any surgical nail of any shape or customized to particular surgical nail systems with bespoke mountings etc. to ensure the correct nail is used for a particular situation.

Prior placement of distal locking screws was done primarily by a variety of freehand techniques, using conventional or radiolucent drilling devices. These methods may result in repeated drilling, repeated X-Ray adjustment, which may require longer exposure as well as higher potential for mis-drilling. This is to be avoided so the present inventions effectively pre-calibrate an arrangement for positioning a nail and a locator to provide adjustment for installation distortion.

The inventions can be adapted to determine a targeting position on a surgically implantable nail adapted to be used in internal fixation of a long bone, the nail comprising a distal end and a proximal end. Ideally the inventions are provided in a device which includes an articulating arm that is adapted to be detachably coupled to the proximal end of the nail, this arm is rotationally and longitudinally adjustable to be compatible with any nail of any shape then locked in that position, and a locator jig for targeting adjustably coupling to the distal end of the arm with a positioning block and a guide with a rod marked in use to provide subsequent return to relative position between them for drilling.

A combined invention device consists of three main parts:
1—An extendable first part typically in the form of a telescopic arm in accordance with the first invention or a pivot articulated arm in accordance with the second invention and a universal angular adjustment means which typically includes two multidirectional ball and socket type joints. The proximal end of the arm is attached firmly by a mounting or clamp to a nail inserting handle using pegs and screws or any other method. A distal end of the arm is attached to a second part of the device below. The telescopic articulating/pivot articulation function and the ball and socket joints of the arm allows it to be used with any surgical nail of any given length or shape, is very strong and doesn't become loose during device handling intra-operatively between stages of nail insertion and locking as described later.
2—A second part is provided by a targeting jig or positioning block and is attached firmly to the extendable first part (the telescopic arm). The jig or block contains holes or apertures that match the axes and direction of the nail holes or apertures in the surgical nail in order to guide a drilling device in to the bone and through in to the nail holes or apertures as necessary. The block in accordance with preferred aspects of a locator has adjustment through a sliding function which enables the jig or block holes/apertures to follow the nail deformation during insertion and so the nail holes/apertures as well. This second part can also be combined or possibly replaced with use of a target device as described below with regard to a fourth invention.
3—A third part is provided by a calibration arch of the first invention. The third part is attached firmly to the second part and consists of an L shaped arch have a guide generally in the form of a sleeve at the free or distal end of the arch and a calibrating rod which identifies through a marker the surgical nail position within the bone during a pre-installation phase and allows subsequent return to that relative positioning upon installation of the surgical nail in to the bone.

The method of installation includes the following steps:
A—Check that the targeting jig or block is free and is in the neutral position to allow adjustment.
B—Calibration of distal locking holes must be done prior to surgical nail insertion into a bone so by using positioning pins which extend from the jig or block to the nail apertures in the nail so that an acceptable relative position is achieved and then secured with nuts or other means so that the extendable telescopic/universal joint arm is locked in position.
C—Once in an acceptable relative position it is important to ensure that the positioning pins slide smoothly in the holes of the nail so that the locking screws to lock the distal end position of the nail will also be easily located with limited stress.
D—Adjustment of the calibrating arch which accommodates a rod which is guided all the way to the anterior nail surface by a guide in the form of a sleeve upon the arch. In this manner, the calibrating arch will extend over the surgical nail so that the rod can be marked in some way for recording relative position. This mark is a specific reading upon the rod and normally takes a semi-permanent form through an ink mark or clamped on retainer. The position where the rod and in particular a foot end to the rod engages the surface of the surgical nail will change if the nail deforms. Such deformation will be seen through the marker or reading as the rod can freely or controllably side in the guide sleeve. Normally there will be means to adjust the positioning block by a sliding mechanism which will allow movement of the block so that with the calibration arch secured to the block the locator can be configured so that the displaced marking or reading is returned to be consistent with the original marking position for subsequent return of relative position for consistency between the relaxed state and the inserted state to enable the drill and locking screws to be inserted and secured in the correct position without the need for X ray guidance.
E—Once the surgical nail is ready to be positioned, located and locked the positioning arrangement including the locator jig are removed from the nail. The nail is then inserted into the typically reamed bone intramedullary canal.
G—Once the nail is forced into the bone intramedullary canal then the device normally comprising the positioning arrangement and the locator respectively with the positioning arm and the calibrating arch is reattached to the nail at the open or proximal end.
H—The guide of the locator is then used to drill down through the bone to the surgical nail.
I—The rod is then located in the guide and slid down until there is contact with the nail and review of marker position made so there can be adjustment of the distal positioning block until the pre-calibrated relative positioning is achieved that is to say the marker position is the same for the pre-calibrated condition and the inserted condition. Thus with the positioning arrangement comprising the positioning arm with extension means and universal joints locked and the markers consistent the position apertures in the position block should be substantially aligned with the nail apertures so a drilling device can be guided through the positioning block will coincide with the nail apertures.
J—Nail distal locking is now achieved by drilling an aperture in the bone and inserting a locking screw appropriately. This aspect can be further enhanced at least by using a target device in accordance with fourth aspects of the invention where an irradiation image is taken and effectively calibrated so a target line can be aligned with a feature of the nail.
   Typically there are three options for distal locking of a surgical nail:
1—Static locking: One screw is placed in a round hole and the other is placed in the proximal part of an oblong hole in the nail with apertures drilled appropriately thorough the bone.
2—Dynamic locking: Locking in the distal part of an oblong hole in a surgical nail creates a “Dynamic Locking” (slide) mechanism—requires only one screw
3—Static/dynamic locking: one screw is placed in the distal part of the oblong hole and the other in the round hole in a surgical nail. If dynamization is required after a period of time, the screw that was placed in the round hole is removed to allow a dynamic locking action as described above with regard to option 2.
   All these 3 options are achievable with the present invention.

FIG. 1 provides a schematic perspective view of a device incorporating a locator jig 1 in accordance with aspects of the first invention and a positioning arrangement 2 in accordance with aspects of the second invention.

The locator 1 comprise a positioning block 3 secured to a distal end of a positioning arm 4. The positioning block 3 may be a solid block of robust material as depicted or another structure to provide positioning apertures 5 each arranged to be consistent with distal end surgical nail apertures or holes 6 in a surgical nail 8. It will be understood that the block 3 may be generic or specific blocks provided for specific or groups or particular manufacturers of nails in terms of aperture sizes, spacing, orientation and number.

Associated with the block 3 is a calibration arch 7 which is generally L shaped to extend over the surgical nail 8 with a guide 9 positioned to guide and project a rod 10 with a foot end 11 towards the nail 8.

In use the rod 10 slides along or through the guide 9 typically in the form of a sleeve or open sided channel or is captive on a rail until the foot end 11 engages an anterior surface of the nail 8. This engagement position is marked in some way on the rod 10 or an associated part and typically by a semi-permanent pen ink or clamp marker/retainer or otherwise. Alternatively, the rod 11 may have accurate incremental numbering or markings which can be recorded for reference later as a calibration marker. Such marking in use only occurs when a respective positioning pin (not shown) is located and preferably secured between the at least one and preferably all the holes 5 in the block 3 and at least one and normally all the apertures 6 in the nail 8. The marking or reading provides a reference for relative positioning between the block 3 and the holes 6 and the nail 8.

Once marking of the rod or an associate part has occurred to register the rod 10/end 11 and so nail relative position, the rod 11 can be removed and put in a safe location. The nail 8 can then be inserted in accordance with normal practice or as described below with regard to the second invention within a bone (not shown). The nail 8 may distort during the insertion process. Thus, when the arm 4 is used to present the block 3 again then when the rod 10 with the end 11 are presented through the guide 9 it will be appreciated that the marking on the rod 11 or an associated part may not be consistent with the pre-calibration marking position prior to insertion. In such circumstances the block 3 includes an adjuster comprising a frame 12 and a screw thread displacement mechanism so that a carrier part upon which the arch 7 is secured can be adjusted until the marked position upon the rod 10 is returned and alignment of the positioning holes 5 and the nail holes 6 is achieved. Once aligned apertures can be drilled and more permanent pins or screws arranged to extend in to the apertures to secure the bone to the nail 8.

It will be understood to provide a reference or calibration for installation of a surgical nail means must be provided to accommodate not only for each type and style of nail but also for differences in manufacturer and tolerance variation. A second invention which typically will be combined with the first invention but not necessarily compromises an arm 4 which has longitudinal extension means such as a telescopic section 14 with at least two universal joints such as ball and socket joints 15, 16. An alternative to the second invention is the third invention described below but some aspects of the description apply to both inventions. The arm 4 is secured by a mounting 17 to a handle 18 which in turn is secured on the other side to a surgical nail 8. In use the nail 8 and the arm 4 are broadly parallel to each other.

In order to calibrate the nail 8 and the arm 4 assembly the extension means 14 and universal joint(s) 15, 16 are released and allowed to freely adjust as the block 3 and distal end of the nail 8 are associated in an arrangement where one or normally all the positioning apertures 5 and the nail apertures 6 are connected and secured by pins or screws (not shown). In this state the extension means 14 and universal joints 15, 16 adjust for curvature, shaping and otherwise for the actual nail 8 in use rather than a notional design standard. The extension means 14 and the universal joints 15, 16 are locked in the adjusted state. It will be understood that the surgeon can also ensure that the actual pins, screws or bolts to be used to fix the nail 8 at the distal end to bone fit and assemble perfectly. This condition is stabilised by locking the extension means 14 and the joints 15, 16. The locks used may be of any suitable type including compression, friction and interference fits.

For installation of a nail 8 typically the handle 18 will be detached from the nail 8 through release of a retainer mounting 19. The pins extending between the positioning block 3 and the distal end of the nail 8 are also removed so that the remainder of the arrangement can be set aside in its locked state. The nail 8 can then be inserted into the bone as required then the handle and remainder to the arrangement re-attached with drilling to the nail apertures as previously described.

FIG. 2 provides basic schematic illustrations of the stages a) to d) of surgical nail installation within a bone 20 with a fracture 21. Thus, at stage a) the fracture 21 is reviewed and set in the desired restored state so that in accordance with aspects of the present invention a clinician or surgeon can determine a surgical nail is needed and determines the length Z and type required. Between stages a) and b) the processes of calibration in accordance with the first invention and the second or third invention above are performed such that at stage b) a surgical nail 23 such as an IMN is inserted after incisions and reaming etc. In such circumstances it is necessary to find the nail apertures 22 for distal end locking in the bone 20. At stage c) the positioning arrangement and/or the locator as described above are used to position a drill device to drill holes to align with the nail apertures 22 and to adjust for nail distortion upon insertion to the bone 20. At stage d) the locking screws are inserted and secured to stabilise and lock the distal end of the nail 23. A target device as described can also be used for drilling and/or screw positioning.

As described above with the locator the end foot will be allowed to slide upon the rod through the guide to contact the nail 23 surface. Adjustment to the marker made during calibration can be made for subsequent return to relative positioning between a positioning block 24 and the distal end of the nail or more particularly the nail holes 22 and positioning holes 25. Thus, a drilling device presented through the holes 25 will drill in substantive alignment with the nail holes or apertures 22. The foot end of the marker may extend through a hole drilled into the bone through the guide to provide access to the nail surface for positioning.

As described above, with a positioning arrangement 26 comprising an adjustable (extension and angular) arm 27 locked in the desired configuration when the arrangement 26 is secured to the nail 23 again then the positioning block 24 should again return to the desired relative position between the block 24/distal end of the nail 23 or holes 22, 25 ignoring nail installation distortion but normally some adjustment will be required for such distortion. Thus, at stage d) the device described above is re-attached to the nail 23 so that the holes 22, 25 are aligned and so that drilled holes can be formed for locking the distal end of the nail to the bone.

FIG. 3 provides a schematic illustration of a locator in accordance with aspects of the first invention. Thus, as previously the objective is to align the nail holes 6 with positioning holes 5 in a positioning block 3 secured to an arm 4 so that holes through bone 30 can be drilled and subsequently screws used to lock the distal end of the nail 8. The locator has an arch 7 extending from the block 3 over the nail 8 such that the foot end 11 can engage the nail to determine distortion and provide adjustment of the block 3 accordingly to provide alignment. The foot end 11 is on a rod 10 which extends through the guide 9 so that as described above a marker can be provided on the rod 10 itself or another part by proxy for relative positioning to give a semi-permanent reference on the rod or by recordal of incremental position for subsequent return. In any event by using the calibration marker as a reference and consistency between pre and post installation it will be understood that alignment of the positioning holes 5 for a drilling device and the nail holes 6 is more assured. This assurance is further confirmed by use of a targeting device as described below.

A specific further hole for the foot end 11 and rod 10 can be drilled to gain access to the nail 8. The block 3 is generally at least adjustable by appropriate means perpendicular to the illustration of FIG. 3 but may have other domains of adjustment dependent upon the need to achieve return to the marker position determined upon pre installation calibration. By such an approach greater adjustment of the positioning block for consistency with distortions of the nail can be provided.

It will be understood that the arch and guide will tend to be reusable with appropriate sterilisation or disposable, and the rod and foot end either reusable or disposable.

FIG. 4 provides a schematic plan view of a positioning arrangement in accordance with aspects of the second invention. Thus, as previously an adjustable arm 4 is secured at one (proximal) end to a handle 18 to which a nail 8 is also secured. At the respective distal ends of the arm 4 and the nail 8 during calibration pins 40 extend across between a positioning block 3 and the distal end of the nail 8 thorough respective apertures 5, 6. Once assembled the positioning arrangement is locked by locking the extension means and the universal joint means in the arm 4. The handle 18 is then detached from the nail 8 and the nail 8 driven into a bone as described previously. If there is no distortion of the nail then when the nail 8 now inserted into the bone is re-attached to the handle and arm/block combination the positioning holes in the block should be returned to substantive alignment with the nail holes or apertures so holes drilled by projection from the positioning holes in the block will align with the nail holes to allow locking screws or bolts to be secured by drilling there through bone to underlying nail apertures.

FIG. 5 provides a schematic illustration of a third invention with respect to a positioning arm 50 in accordance with aspects of the present invention. As previously a surgical nail 51 is secured by a clamp 52 at one proximal end and a positioning jig or block 53 at the other distal end. Thus, the positioning arm 50 comprises a clamp part 54 and a distal part 55 which is articulated about a pivot 56 in the form of a hinge. By a scissor effect A about the pivot 56, a distance 57 between the clamp 52 at a clamp end 58 and the jig 53 at the distal end 59 can be varied as required for the positioning of the nail 51 upon the arm 50.

The distance 57 is variable and set by a cross-member 49 through a fastener or fasteners which are secured between the parts 54, 55 typically with a positionally fixed end 48 and a positionally variable end 47 with a fastener such as a clamping bolt 46. Normally the fixed end 48 and the variable end 47 will be substantially the same distance from the pivot 56 for balance and stability. It will be noted that the cross-member 49 and a line of minimum distance 57 spacing the ends 58, 59 are generally parallel. The parts of the arm 50 are normally formed and made from materials to allow sterilisation and possibly of a surgical grade steel so precise operation is achievable.

The fixed end 48 will generally be provided by a hinge pin extending through the part 54 and an end of the cross-member 49. A number of holes (not shown) may be provided in the part 54 so that the fixed end 48 can be provided at a number of positions. It will also be understood in some situations it may be desirable to provide a positionally variable cross member end to both parts 54, 55 through appropriate bolt fasteners or otherwise. The cross-member 49 may be rigid in terms of length or some form of fine adjustment allowed allowing micro changes for fine positioning or the cross-member may allow slight distortion in preference to overly stressing the process of drilling or locating pin insertion in to a nail aperture.

As a feature of the third invention is variability in the distance 57 it will be understood that providing a fastener at the end 47 is important. As illustrated this is typically by a slide channel 45 with a clamp bolt 46 so that the cross-member 49 can be associated and locked with regard to the parts 54, 55 as desired. Essentially, the parts 54, 55 will be articulated until the desired distance 57 is achieved with the bolt 46 sliding in the channel 45 then the bolt 46 clamped to secure the cross-member 49 position and so the distance 57. It will be understood that normally as indicated above universal joints 44, 43 will also be provided to allow positioning of the clamp and the positioning jig or block as required for an associated nail 51 in use.

Clearly if would be desirable for the positioning arm to be integral but provision of a detachable cross-member might be acceptable for cleaning and to allow different sized cross-arms for different conditions and nails. It will also be understood rather than initiating adjustment from first positions each time the cross-member may be integrally marked for matching with surgical nails as presented with fine adjustment then provided by the movement about the fastener 46 or the cross-member designed to allow marking/be markable so allowing repeating of that position later upon use with the surgical nail inserted.

Normally the cross-member as illustrate will be straight but in some circumstances a curved cross-member may be used to provide a different variation and adjustment range for the positioning arm. The cross-member will normally be rigid but if desired some flexibility/malleability may be provided for adjustment in use.

As indicated above traditionally a C arm irradiation source and image target have been provided to finely determine the position of a distal end of a surgical nail such as an intra-medullary nail (IMN) in terms of finding features such as holes/aperture for drilling bone and positioning of securing pins etc. Clearly the angle of the nail, irradiation or X-ray source and image target are all important with regard to correct determination of position. FIG. 6 and FIG. 7 illustrate a target device 60 to facilitate calibration and good positioning or accurate determination by ensuring initially a level association is achieved and then using a fixed target line as a reference for the comparison with a feature of the nail for positioning and presentation during drilling and positioning of pins.

The target device 60 comprises an irradiation clear housing 61 with a plurality of, usually two, opaque line elements 62, 63 in an irradiated image in spaced parallel positions in a plane and a clearly displaced target line element 64 with a displacement spacer 65 between them. The positioning process thus does not obscure the view of target line over lay with a nail feature such as an aperture in an irradiation image. As the housing 61 is irradiation clear the elements 62, 63, 64 will be seen separately and distinctly in an irradiation image. The elements 62, 63 will typically be formed of thick metal wire of a few millimetres thickness and the target line element of metal wire a millimetre or more in diameter provided the element can be seen in an irradiation X-ray image. The housing 61 will normally be made from an irradiation clear material such as a plastics material so that the elements 62, 63, 64 will appear to ‘float’ in the image as provide by a conventional C-arm X-Ray irradiation arrangement used for positioning as depicted in FIG. 8. It will be understood that the opaque line elements 62, 63 will be aligned in a flat plane parallel with the line of the irradiation source to image device.

In FIG. 8 a target device 60 as described above with regard to FIGS. 6 and 7 is provided in a C-arm arrangement with an X-ray source 66 on one side and an image device 67 on the other side of the C-arm X-ray machine arrangement. In the illustration of FIG. 8 the target 60 is located in between the source 66 and the image device 67 along with the representative hollow bone 68 with a nail 69 having a hole 70 for a fixing pin (not shown). In use the target 60 is mounted on a positioning arm with the target 60 allowing correct positioning for drilling and/or inserting of a fixing pin for secure a surgical nail.

As can be seen in FIG. 8 the opaque elements 62, 63 are spaced, parallel and in a plane so that if the target 60 is not parallel as illustrated in image a) two lines will be seen from the elements 62, 63 but if parallel between the source 66 and image device 67 then the image b) will show only one line with the opaque elements 62, 63 in the image casting shadows which coincided and respectively overlap to form a single line. The target line element 64 is a single element so will always be a single line in the image which if the opaque elements 62, 63 are aligned can act as a reference for matching with features within a nail such as a hole. The target line element 64 need not be in the plane of the irradiation source 66 and image device 67 as there is a known stable displacement element so the presentation is also known and predictable.

The opaque line elements 62, 63 in the target are typically two solid and continuous lines of irradiation opaque material such as a metal wire. Clearly, if the lines are of a known length when over layered exactly then that length will also be seen in the image if parallel and perpendicularly in front but if parallel and slightly at an angle this may not been seen other than in a slightly longer or shorter image line. In FIG. 9 several possibilities for opaque line element options are illustrated:
a) Illustrates the normal approach of two solid lines 62, 63 as illustrated above with regard to FIGS. 6 to 8 with i) showing before positioning in a plane and ii) when aligned in a plane;
b) Illustrates interrupted or broken lines for respective opaque elements 162, 163 so that when parallel and aligned perpendicularly to an irradiation source should provide a solid line 100;
c) Illustrates wavy opaque elements 262, 263 which only when parallel and aligned perpendicularly will produce a single solid wavy line 200 rather than shadowy waves side by side and/or above/below alignment.
d) illustrates opaque elements 362, 363 with upstanding elements 101 which again will produce a single image 300 if aligned but have shadowy images when not both if not parallel and perpendicular;
e) illustrates opaque elements 462, 463 which provide end upstanding elements 102 and other distinctive features which when parallel, aligned and perpendicular will provide a solid line 400 but when not will be shadowy.
Once the opaque line elements have shown parallel orientation as indicated for calibration then the target line will be used as a reference for location of the target relative to a feature on a surgical nail. The target will be mounted on a positioning arm using a mounting such as pegs. Thus, once the target device is removed a positioning jig can be re-mounted on the positioning arm so that the jig can then first drill and then place securing pins in apertures ‘found’ using irradiation imaging of the target line element to position it relative to the desired feature of the surgical nail such as the fixing aperture or hole in the nail. The initial confirmation of a parallel relationship using overlap of the opaque line elements of the target device means that the degrees of freedom for deviation with the reference target line are reduced and more predictability provided reducing the number of radiation/X-ray exposures needed.

In FIG. 10 three potential images of the target line 64 relative to a hole or aperture 65 in the nail as seen in an irradiated image are provided. Thus, in FIG. 10a and FIG. 10b it will be noted that the target line 64 image is respectively below ideal centre and above ideal centre of the nail hole or aperture 65 whilst in FIG. 10c the target line 64 image is in the centre of the aperture 64 so that when the target device is removed and the matched positioning jig is returned to the positioning arm the drill or pin location will be substantially aligned with the aperture 65. Adjustments from the unacceptable below and above conditions in FIG. 10a or FIG. 10b is made by adjustment to the positioning arm until the reference target line element 64 image is at the condition as depicted in FIG. 10c and these adjustments are retained and locked in when the positioning jig or block is re-associated with the positioning arm as described above.

For the avoidance of doubt each of the four inventions described above can be provided separately or in respective combination of less than all inventions in one device or all combined together in one device.

It will be appreciated by those skilled in the art that any number of combinations of the aforementioned features and/or those shown in the appended drawings provide clear advantages over the prior art and are therefore within the scope of the invention described herein.

1-53. (canceled)

54. A locator for a surgical nail, the locator comprising a positioning block for surgical fixings and lockable upon a positioning arm spaced in use relative to a surgical nail, the positioning block having an arch capable of extending towards a surgical nail, the arch having a guide and a rod guided by the guide, the rod having a contact end to engage a surgical nail when projected through the guide and capable of indicating the rod position upon engagement with the surgical nail for subsequent return of relative positioning between the positioning block and a surgical nail.

55. A locator as claimed in claim 54 wherein the positioning block comprises a carrier incorporating each positioning aperture in a frame whereby the carrier is moveable in the frame for subsequent return of relative positioning between the positioning block and a surgical nail.

56. The locator of claim 54 where the guide is a sleeve.

57. The locator of claim 56 where the sleeve extends substantially perpendicularly at a spaced position from the positioning block.

58. The locator of claim 54 where the contact end comprises an off-center foot upon the rod.

59. The locator of claim 56 where the rod includes marked increments to allow recording of rod position relative to part of the guide and to allow incremental adjustment.

60. A method of locating a surgical nail in a bone, the method comprising:
a) prior to insertion of a surgical nail into a bone cavity, associating the surgical nail with an arm having a positioning block adjacent a distal end of the nail;
b) securing the block towards the distal end of the surgical nail thorough nail apertures;
c) positioning a calibration arch with a guide over a part of the distal end of the surgical nail, placing within the guide a rod with a foot end to engage a surface of the surgical nail and marking a marker position of the rod upon the rod or an associated part when the foot end engages the surface of the surgical nail with a calibration mark or recording that position;
d) detaching at least the block from the distal end of the nail and inserting the nail into a bone cavity;
e) re-attaching together as required the now inserted surgical nail in the bone cavity and arm as previously with the calibration arch extending over the nail; and,
f) inserting the rod and providing the foot end in engagement with the surface of the surgical nail and adjusting the block to return to the calibration marker or position recorded if required.

61. The method of claim 60 where the positioning block is used to drill an aperture and/or position a fixing such as a pin or screw.

62. A distal lock positioning arrangement for a surgical nail, the arrangement comprising a mounting to present a nail with a distal end having a nail aperture, a positioning arm associated with the mounting by a universal joint, the arm having extension means and at a distal end there is a positioning aperture whereby in use a pin can extend through the positioning aperture to a nail aperture in a surgical nail and at least when suitably aligned the universal joint and the extension means are lockable to allow removal and return of the nail if required in use from attachment to the distal lock positioning arrangement.

63. The arrangement of claim 62 where the universal joint comprises a number of universal joint elements intermediate along the length of the arm.

64. The arrangement of claim 62 where the positioning arm is adjustable at least along an axis in use parallel to a surgical nail.

65. The arrangement of claim 62 where the extension means comprises a telescopic part or means to allow arms of different length to be attached as required for extension.

66. The arrangement of claim 62 where the positioning arm has a first part and a second part pivotally connected to one another, with a clamp proximate an end of a selective one of the first part and the second part, and including a cross-member between the first part and the second part, with a fastener securing the cross member to at least one of the first part and the second part, where the fastener is capable of adjustably fixing the angle of the first part relative to the second part.

67. The arrangement of claim 66 wherein the cross-member is secured to the first part and the second part at substantially the same distance from the pivotal connection between the first part and the second part.

68. An arrangement as claimed in claim 66 wherein one end of the cross member is positionally fixed and the fastener is selectively slidable on the cross-member.

69. A target device for a surgical nail using irradiation, the target device comprising a plurality of irradiation opaque line elements and an irradiation target line element with a displacement element between them, the target device having a mounting for securing the target device to a positioning arm whereby substantially exact coincidence of the opaque line elements one upon the other in an irradiation image provided by an irradiation source to one side defines alignment of the target device and the irradiation source, the displacement element provides the target line element as a reference a clear displaced distance away from the opaque line elements for association with features in an image of a surgical nail feature in use.

70. A target device as claimed in claim 69 wherein the target device is provided in a housing formed from an irradiation clear material to present the opaque line elements and the target line element from the displacement element.

71. A target device as claimed in claim 69 wherein the opaque line elements are continuous and solid or are formed from segments to provide a broken line separately but a continuous line when overlapping in alignment.

72. A target device as claimed in claim 69 wherein the opaque line elements are straight or wavy and/or have upright portions.

73. A target device as claimed in claim 69 wherein the opaque line elements are parallel and separated in a displaced plane relative to the target line element and/or is straight or shaped to interact with parts of a surgical nail in use.
